SEQUENCE LISTING (4 sequences)

SEQ ID NO: 1 - protein, 107 aa, Artificial Sequence [sequence omitted]

SEQ ID NO: 2 - protein, 116 aa, Artificial Sequence [sequence omitted]

SEQ ID NO: 3 - protein, 214 aa, Artificial Sequence
  Synthetic construct
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2019/0336599 A1
(43) Pub. Date: Nov. 7, 2019

(54) COMBINATION OF RAMUCIRUMAB AND MERESTINIB FOR USE IN TREATMENT OF COLORECTAL CANCER

(71) Applicant: Eli Lilly and Company, Indianapolis, IN (US)

(72) Inventors: Sau-Chi Betty Yan, Indianapolis, IN (US); Richard Anthony Walgren, New Palestine, IN (US)

(73) Assignee: Eli Lilly and Company, Indianapolis, IN (US)

(21) Appl. No.: 16/093,255

(22) PCT Filed: Apr. 7, 2017
(86) PCT No.: PCT/US2017/026552

§ 371 Date: Oct. 12, 2018

Related U.S. Application Data
(60) Provisional application No. 62/323,269, filed on Apr. 15, 2016.

(51) Int. Cl. A61K 39/395; A61K 31/4439; C07K 16/28

(52) U.S. Cl. CPC A61K 39/39541; C07K 2317/21; C07K 16/2863; A61K 31/4439

(57) ABSTRACT

The present disclosure relates to a combination of an anti-human VEGFR2 antibody, preferably ramucirumab, and merestinib, or a pharmaceutically acceptable salt thereof, and to methods of using the combination to treat certain disorders, such as colorectal cancer, including advanced or metastatic colorectal cancer and/or local colorectal cancer.

The present invention relates to a combination of an anti-human VEGFR2 antibody, preferably ramucirumab, and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (merestinib), or a pharmaceutically acceptable salt thereof, and to methods of using the combination to treat certain disorders, such as colorectal cancer, including advanced or metastatic colorectal cancer and/or local colorectal cancer.

Dysregulation of the MET signaling pathway occurs in a wide range of human cancers, including the most common epithelial cancers such as colorectal cancer (Peters et al., Nature Rev, Vol. 9, 2012). Overexpression of RON in primary tumors such as colon cancer is predictive of patient survival and correlates with clinical and pathological parameters (Yao et al., Nature Rev, Vol. 13, 2013). Primary colorectal cancers and their subsequent hepatic metastases have been found to be genetically different, with variations found, for example, in genes such as KRAS, BRAF, KDR, and PI3KCA and in the up/downstream genes of EGFR/PI3K/VEGF-pathways. Thus, genetic properties of the metastasis instead of those of the primary tumor should provide bases for treatment choice (Vermatt et al., Clin Cancer Res, 18(3), 2012). Also, MKNK1/2 activity and its substrate EIF4E are found to be involved in the malignant transformation of colorectal cancer (Fan et. al, Cancer Biol Ther, Vol. 8, 2009; Hou el. al., Oncotarget, Vol. 3, 2012; De Benedetti et. al., Oncogene, Vol. 23, 2004).

N-(3-fluoro-4-(1 -methyl-6-(1H-pyrazol-4-yl)-1H-indazol-5-yloxy)phenyl)-1-(4-fluorophenyl)-6-methyl-2-oxo-1,2-dihydropyridine-3-carboxamide,

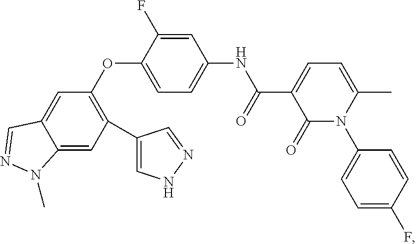 [SMILES: Cc1ccc(C(=O)Nc2ccc(Oc3cc4cnn(C)c4cc3-c3cn[nH]c3)c(F)c2)c(=O)n1-c1ccc(F)cc1]

also referred to as LY2801653 or merestinib, or a pharmaceutically acceptable salt thereof, is active against, for example, MET and RON, as well MKNK1/2 (Yan et al., Invest New Drugs, Vol. 31, 2013). Merestinib, or a pharmaceutically acceptable salt thereof, and methods of making and using this compound including for the treatment of cancer and more specifically for the treatment of colorectal cancer are disclosed in WO2010/011538. Furthermore, merestinib is currently being investigated in a Phase I clinical trial for advanced cancer in the United States (A Phase 1 Study of LY2801653 in Patients With Advanced Cancer, NCT01285037). One objective of this study is to determine a recommended Phase 2 dose of merestinib that may be safely given to participants with gastric cancer when taken with ramucirumab.

Ramucirumab (CYRAMZA®) is a fully human monoclonal antibody directed against the vascular endothelial growth factor receptor 2 (VEGFR2). Ramucirumab and methods of making and using this compound including for the treatment of neoplastic diseases such as solid and non-solid tumors are disclosed in WO2003/075840. Ramucirumab is approved by the U.S. F.D.A. as a single agent, or in combination with paclitaxel, for the treatment of patients with advanced or metastatic gastric or gastroesophageal (GE) junction adenocarcinoma with disease progression on or after prior fluoropyrimidine- or platinum-containing chemotherapy; in combination with docetaxel, for the treatment of patients with metastatic non-small cell lung cancer (NSCLC) with disease progression on or after platinum-based chemotherapy; and in combination with FOLFIRI (irinotecan, folinic acid, and 5-fluorouracil) chemotherapy, for the treatment of patients with metastatic colorectal cancer (mCRC) with disease progression on or after prior therapy with bevacizumab, oxaliplatin, and a fluoropyrimidine.

Broadly applicable therapies for cancer, in particular colorectal cancer, including advanced or metastatic colorectal cancer and/or local colorectal cancer, still remains elusive and, thus, there exists a need for more and different therapies that may prove to be effective in treating advanced or metastatic colorectal cancer and/or local colorectal cancer.

A clinical trial investigating the combination of LY2801653 and ramicirumab for the treatment of gastric cancer has been disclosed (NCT01285037). However, the present invention discloses herein methods for treating colorectal cancer, including advanced or metastatic colorectal cancer and/or local colorectal cancer, in a patient that provides enhanced and/or unexpected beneficial therapeutic effects from the combined activity of merestinib, or a pharmaceutically acceptable salt thereof, and ramucirumab as compared to the therapeutic effects provided by either agent alone. Furthermore, the present invention discloses methods for treating colorectal cancer, including advanced or metastatic colorectal cancer and/or local colorectal cancer, in a patient as part of a specific treatment regimen that provides enhanced and/or unexpected beneficial therapeutic effects from the combined activity of merestinib, or a pharmaceutically acceptable salt thereof, and ramucirumab as compared to the therapeutic effects provided by either agent alone. More particularly, use of this combination may provide enhanced and/or unexpected beneficial therapeutic effects to both KRAS positive and KRAS negative patients.

Accordingly, the present invention provides a method of treating colorectal cancer in a patient, comprising administering to a patient in need of such treatment an effective amount of an antibody comprising a light chain variable region (LCVR) amino acid sequence of SEQ ID NO: 1 and a heavy chain variable region (HCVR) amino acid sequence of SEQ ID NO: 2, wherein the antibody binds to VEGFR2, and a compound which is merestinib, or a pharmaceutically acceptable salt thereof. In a preferred aspect of the invention, the antibody comprises a light chain amino acid sequence of SEQ ID NO: 3, and a heavy chain amino acid sequence of SEQ ID NO: 4 and the antibody binds to VEGFR2. In another preferred aspect of the invention, the antibody is ramucirumab. In a further preferred aspect of the invention, the compound or salt thereof, is administered at a dose of about 80 mg once every day of a 28-day cycle and ramucirumab is administered at a dose of about 8 mg/kg on Days 1 and 15 of a 28-day cycle.

The present invention also provides a kit comprising an antibody comprising a light chain variable region (LCVR) amino acid sequence of SEQ ID NO: 1, and a heavy chain variable region (HCVR) amino acid sequence of SEQ ID NO: 2, wherein the antibody binds to VEGFR2, and a compound which is merestinib, or a pharmaceutically acceptable salt thereof, for simultaneous, separate, or sequential use in the treatment of colorectal cancer. In a preferred aspect of the invention, the antibody comprises a light chain amino acid sequence of SEQ ID NO: 3, and a heavy chain amino acid sequence of SEQ ID NO: 4, wherein the antibody binds to VEGFR2. In another preferred aspect of the invention, the antibody is ramucirumab.

The present invention further provides a kit comprising ramucirumab, with one or more pharmaceutically acceptable carriers, diluents, or excipients, and a compound which is merestinib, or a pharmaceutically acceptable salt thereof, with one or more pharmaceutically acceptable carriers, diluents, or excipients, for simultaneous, separate, or sequential use in the treatment of colorectal cancer. In a preferred aspect of the invention, the compound or salt thereof, is provided in the form of a tablet. In another preferred aspect of the invention, the tablet is formulated by spray dried dispersion.

The present invention additionally provides a combination comprising an anti-VEGFR2 antibody and a compound which is merestinib, or a pharmaceutically acceptable salt thereof, for simultaneous, separate or sequential use in the treatment of colorectal cancer. In a preferred aspect of the invention, the anti-VEGF antibody comprises a light chain variable region (LCVR) amino acid sequence of SEQ ID NO: 1 and a heavy chain variable region (HCVR) amino acid sequence of SEQ ID NO: 2. In another preferred aspect of the invention, the anti-VEGF antibody comprises a light chain amino acid sequence of SEQ ID NO: 3, and a heavy chain amino acid sequence of SEQ ID NO: 4. In yet another preferred aspect of the invention, the anti-VEGF antibody is ramucirumab. in a further preferred aspect of the invention, the compound or salt thereof is administered at a dose of about 80 mg once every day of a 28-day cycle and ramucirumab is administered at a dose of about 8 mg/kg on Days 1 and 15 of a 28-day cycle.

The present invention also provides an anti-VEGFR2 antibody for simultaneous, separate or sequential use in combination with a compound which is merestinib, or a pharmaceutically acceptable salt thereof, in the treatment of colorectal cancer. In a preferred aspect of the invention, the anti-VEGF antibody comprises a light chain variable region (LCVR) amino acid sequence of SEQ ID NO: I and a heavy chain variable region (HCVR) amino acid sequence of SEQ ID NO: 2. In another preferred aspect of the invention, the anti-VEGF antibody comprises a light chain amino acid sequence of SEQ ID NO: 3, and a heavy chain amino acid sequence of SEQ ID NO: 4. In yet another preferred aspect of the invention, the anti-VEGF antibody is ramucirumab.

The present invention also provides a compound which is merestinib, or a pharmaceutically acceptable salt thereof, for simultaneous, separate or sequential use in combination with an anti-VEGFR2 antibody in the treatment of colorectal cancer. In a preferred aspect of the invention, the anti-VEGF antibody comprises a light chain variable region (LCVR) amino acid sequence of SEQ ID NO: 1 and a heavy chain variable region (HCVR) amino acid sequence of SEQ ID NO: 2. In another preferred aspect of the invention, the anti-VEGF antibody comprises a light chain amino acid sequence of SEQ ID NO: 3, and a heavy chain amino acid sequence of SEQ ID NO: 4. In yet another preferred aspect of the invention, the anti-VEGF antibody is ramucirumab.

The present invention also provides the use of an antibody comprising a light chain variable region (LCVR) amino acid sequence of SEQ ID NO: 1 and a heavy chain variable region (HCVR) amino acid sequence of SEQ ID NO: 2, wherein the antibody binds to VEGFR2, in the manufacture of a medicament for the treatment of colorectal cancer, wherein the medicament is to be administered simultaneously, separately or sequentially with merestinib, or a pharmaceutically acceptable salt thereof for the manufacture of a medicament for simultaneous, separate or sequential use in the treatment of colorectal cancer. In a preferred aspect of the invention, the compound is merestinib. In another preferred aspect of the invention, the antibody comprises a light chain amino acid sequence of SEQ ID NO: 3, and a heavy chain amino acid sequence of SEQ ID NO: 4 and the antibody binds to VEGFR2. In yet another preferred aspect of the invention, the antibody is ramucirtunab.

Additionally, the present invention provides the use of merestinib, or a pharmaceutically acceptable salt thereof, in the manufacture of a medicament for the treatment of colorectal cancer, wherein the medicament is to be administered simultaneously, separately or sequentially with an antibody comprising a light chain variable region (LCVR) amino acid sequence of SEQ ID NO: 1 and a heavy chain variable region (HCVR) amino acid sequence of SEQ ID NO: 2, wherein the antibody binds to VEGFR2. In a preferred aspect of the invention, the compound is merestinib. In another preferred aspect of the invention, the antibody comprises a light chain amino acid sequence of SEQ ID NO: 3, and a heavy chain amino acid sequence of SEQ ID NO: 4 and the antibody binds to VEGFR2. In yet another preferred aspect of the invention, the antibody is ramucirumab.

Metabolites of merestinib include:

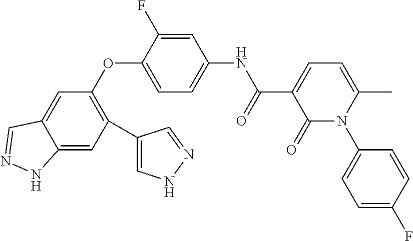 [SMILES: Cc1ccc(C(=O)Nc2ccc(Oc3cc4cn[nH]c4cc3-c3cn[nH]c3)c(F)c2)c(=O)n1-c1ccc(F)cc1]

1-(4-fluorophenyl)-N-[3-fluoro-4- [[6-(1H-pyrazol-4-yl)-1H-indazol-5-yl]oxy]phenyl]-6-methyl-2-oxo-pyridine-3-carboxamide, and,

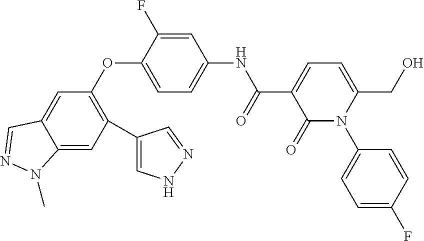 [SMILES: Cn1ncc2cc(Oc3ccc(NC(=O)c4ccc(CO)n(-c5ccc(F)cc5)c4=O)cc3F)c(-c3cn[nH]c3)cc21]

N-[3-fluoro-4-[1-methyl-6-(1H-pyrazol-4-yl)indazol-5-yl]oxy-phenyl]-1-(4-fluorophenyl)-6-(hydroxymethyl)-2-oxo-pyridine-3-carboxamide.

As used herein, the term “VEGFR2” refers to Vascular Endothelial Growth Factor Receptor 2, which is known in the art. VEGFR2 is also known as KDR.

As used herein, the term “anti-VEGFR2 Ab” refers to an antibody comprising: a light chain variable region (LCVR) whose amino acid sequence is that given in SEQ ID NO: 1, and a heavy chain variable region (HCVR) whose amino acid sequence is that given in SEQ ID NO: 2, wherein the anti-VEGFR2 Ab binds to VEGFR2 with sufficient affinity and specificity. In some embodiments, an anti-VEGFR2 Ab is an antibody comprising: a light chain whose amino acid sequence is that given in SEQ ID NO: 3, and a heavy chain whose amino acid sequence is that given in SEQ ID NO: 4 and that binds to VEGFR2 with sufficient affinity and specificity. In other embodiments of the present invention the anti-VEGFR2 Ab is ramucirumab. The antibody selected will have sufficiently strong binding affinity for VEGFR2. For example, the antibody will generally bind VEGFR2 with a K_{d} value of between about 100 nM-about 1 pM. Antibody affinities may be determined by a surface plasmon resonance based assay (such as the BIAcore assay is described in PCT Application Publication No. WO2005/012359); enzyme-linked immunosorbent assay (ELISA); and competition assays (e.g., a radiolabeled antigen binding assay (RIA)), for example. In one embodiment, Kd is measured by a RIA performed with an anti-VEGFR2 Ab, preferably ramucirumab.

As used herein, the term “ramucirumab” also known as CYRAMZA®, IMC-1121b, CAS registry number 947687-13-0, refers to an anti-VEGFR2 Ab comprising: two light chains, each of whose amino acid sequence is that given in SEQ ID NO: 3, and two heavy chains, each of whose amino acid sequence is that given in SEQ ID NO: 4.

Unless indicated otherwise, the term “antibody” or “Ab” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes amino acid sequences of CDRs and FRs.

As used herein, the term “heavy chain variable region” “HCVR” refers to a portion of a heavy chain of an antibody molecule that includes amino acid sequences of CDRs and FRs.

As used herein, the term “kit” refers to a package comprising at least two separate containers, wherein a first container contains merestinib, or a pharmaceutically acceptable salt thereof, and a second container contains an anti-VEGFR2 Ab, preferably ramucirumab. A “kit” may also include instructions to administer all or a portion of the contents of these first and second containers to a colorectal cancer patient.

As used herein, the terms “treating,” “to treat,” or “treatment” refers to restraining, slowing, stopping, reducing, shrinking, maintaining stable disease, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers. Examples of cancer as provided in the present invention include colorectal cancer, including but not limited to particular types of colorectal cancer such as advanced or metastatic colorectal cancer and/or local colorectal cancer.

As used herein, the term “effective amount” refers to the amount or dose of merestinib, or a pharmaceutically acceptable salt thereof, and the amount or dose of an anti-VEGFR2 Ab, preferably ramucirumab, which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of merestinib, or a pharmaceutically acceptable salt thereof, and an anti-VEGFR2 Ab, preferably ramucirumab.

As used herein, the phrase “in combination with” refers to the administration of merestinib, or a pharmaceutically acceptable salt thereof, and an anti-VEGFR2 Ab, preferably ramucirumab, either simultaneously or sequentially in any order, such as, for example, at repeated intervals as during a standard course of treatment for a single cycle or more than one cycle, such that one agent can be administered prior to, at the same time, or subsequent to the administration of the other agent, or any combination thereof.

A main advantage of the combination treatments of the invention is the ability of producing marked anti-cancer effects in a patient without causing significant toxicities or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and/or cell cycle activity, tissue-based biomarkers for angiogenesis and/or cell cycle activity, and measurement of response through radiological imaging.

When given in combination with an anti-VEGFR2 Ab, for example, over a 28-day cycle, merestinib, or a pharmaceutically acceptable salt thereof, is orally administered once every day at a dose of about 40 mg to 120 mg and an anti-VEGFR2 Ab, preferably ramucirumab, is administered on Days 1 and 15 at a dose of about 4 mg/kg to 12 mg/kg. Preferably, ramucirumab is administered by intravenous infusion at a dose of about 4 mg/kg to 10 mg/kg on Days 1 and 15 of a 28-day cycle, more preferably at a dose of about 6 mg/kg, and most preferably at a dose of about 8 mg/kg. Preferably, merestinib, or a pharmaceutically acceptable salt thereof, is orally administered at a dose of about 40 mg to 120 mg once every day of a 28-day cycle, more preferably at a dose of about 40 mg, and most preferably at a dose of about 80 mg. The combination of ramucirumab and merestinib, or a pharmaceutically acceptable salt thereof, is preferably administered simultaneously, separately, or sequentially.

The free base of merestinib, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, is preferred. However, it will be understood by the skilled reader that N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide can react with any of a number of inorganic and organic acids to form pharmaceutically acceptable acid addition salts. Such pharmaceutically acceptable acid addition salts and common methodology for preparing them are well known in the art. See, e.g., P . Stahl, et al., Handbook of Pharmaceutical Salts: Properties, Selection and Use (VCHA/Wiley-VCH, 2002); L. D. Bighley, et al., Encyclopedia of Pharmaceutical Technology, 453-499 (1995); S. M. Berge, et al., Journal of Pharmaceutical Sciences, 66, 1, (1977).

The route of administration may be varied in any way, limited by the physical properties of the drugs and the convenience of the patient and the caregiver. An anti-VEGFR2 Ab, preferably ramucirumab, is preferably formulated for parenteral administration, such as intravenous or subcutaneous administration. Merestinib, or pharmaceutically acceptable salt thereof, is preferably formulated for oral administration, although parenteral administration, including intravenous or subcutaneous administration, can be used. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012).

For example, merestinib may be formulated into a tablet. Such tablet can be made from a composition of 20% merestinib:hydroxy propyl methyl cellulose acetate auccinate (HPMCAS) Medium Grade (M) (HPMCAS-M) Spray Dried Dispersion (SDD). The 20% merestinib:HPMCAS-M SDD is made from a spray solution composition (wt %) containing merestinib (1%), HPMCAS-M (4%) and acetone (85.5%) and purified water (9.5%). Ensure merestinib is fully solubilized in the acetone/water solution before addition of the polymer. Before initiating spray drying to make the SDD composition, visually confirm that the polymer is dissolved. The resulting SDD composition is a 20% merestinib:HPMCAS-M SDD (mg/g) with merestinib (200 mg/g) and HPMCAS-M (800 mg/g). If necessary, the amount of drug substance may be adjusted to take into account the assay of the drug substance. If required to maintain mass balance, the weight of HPMCAS-M may be adjusted according to slight changes in assay of the drug substance. Acetone and purified water are removed during processing to residual levels. The formulation composition may contain, for example, SDD merestinib and other excipients such as diluent (e.g., microcrystalline cellulose and mannitol), disintegrant (e.g., croscarmellose sodium), surfactant (e.g., sodium lauryl sulphate), glidant (e.g., syloid silicon dioxide) and/or lubricant (e.g., sodium stearyl fumarate). The making of the tablet involves spray drying to produce the SDD of merestinib followed by roller compaction and compression into tablets. The tablets are then film-coated with HPMC based color mixture.

An example tablet for a unit formula of merestinib SDD film coated tablet, 40 mg dose strength, is described in Chart 1.

CHART 1
Example Unit Formula of N-(3-fluoro-4-(1-methyl-6-(1H-pyrazol-
4-yl)-1H-indazol-5-yloxy)phenyl)-1-(4-fluorophenyl)-6-methyl-2-
oxo-1,2-dihydropyridine-3-carboxamide (merestinib) Tablets 40 mg

| | | |
|---|---|---|
| | | mg/ |
| Ingredient | Function | Tablet |

Active Ingredient

| | | |
|---|---|---|
| 20% merestinib:80% | Active | 200 |
| HPMCAS-M SDD¹ | | |

Intragranular Excipients²

| | | |
|---|---|---|
| Microcrystalline Cellulose (Avicel PH 102 ®)³ | Filler | 20.19 |
| Mannitol (Pearlitol S D 200 ®) | Filler | 19.50 |
| Croscarmellose Sodium | Disintegrant | 6.50 |
| Sodium Lauryl Sulphate | Surfactant | 3.250 |
| Silicon Dioxide (Syloid 244FP ®) | Gildant | 3.250 |
| Sodium Stearyl Fumarate | Lubricant | 2.440 |

Extragranular Excipients

| Microcrystalline Cellulose (Avicel PH 102 ®) | Filler | 65.00 |
|---|---|---|
| Croscarmellose Sodium | Disintegrant | 3.250 |
| Sodium Stearyl Fumarate | Lubricant | 1.620 |
| Total Core Tablet Weight (mg) | | 325.0 |
| Film Coating Excipients | | |
| Color Mixture Blue 03K105008 | Film Coat | 11.38 |
| Purified Water⁴ | Suspension | — |
| | Vehicle | |

| | |
|---|---|
| Total Coated Tablet Weight (mg) | 336.4 |

¹If necessary, the amount of SDD will be adjusted to take into account of the assay of the dispersion.
²A reasonable variation of ±10% is allowed for each excipient unless otherwise stated.
³To accommodate changes in SDD potency and to maintain the total tablet weight, the weight of microcrystalline cellulose may be adjusted if necessary.
⁴Purified water is removed during processing to residual levels.

### Combinations of Ramucirumab and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide Treatment Reduces Endothelial Cell Sprouting

Measure the in vitro reduction of endothelial cell sprouting by an in vitro cell based assay. Use the assay to measure the effect of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and ramucirumab on endothelial cell sprouting.

HUVECs (Lonza #C2519A) are cultured at 37° C. in 5% CO₂ on culture flasks (Corning #356486) in EBM-2 medium (endothelial growth basal medium, Lonza #CC-3156) supplemented with SINGLEQUOTS™ kit (Lonza #CC-4147), with further fetal bovine serum (FBS) supplement to a final 10% FBS and are used at passages 2-5. Human umbilical vein endothelial cells (HUVECs) are harvested from culture flasks which are rinsed with HYCLONE™ Dulbecco's phosphate-buffered saline (DPBS, Fisher Scientific #SH3026402) followed by TrypLE Express (GIBCO™ #12605-010) and are suspended in 5 mL of warm medium. Viable cell counts are determined using a Vi-CELL™ cell counter (Beckman).

Cultured lung cancer associated fibroblast cells (CAFs, Astrand, 60093A, specially prepared for Lilly) are cultured at 37° C. in 5% CO₂ on Corning culture flasks in fibrobalst growth medium (FBM, Lonza # CC-3131) supplemented with SINGLEQUOTS™ kit (Lonza # CC-4126), with further FBS (HYCLONE™ # SH3061102) supplement to a final 10% FBS, and used at passages 3-7. CAB are harvested from culture flasks which are rinsed with HYCLONE™ DPBS (Fisher Scientific #SH3026402) followed by TrypLE Express (GIBCO™ #12605-010) and are resuspended with 5 ml of warm medium. Viable cell counts are determined using a Vi-CELL™ cell counter (Beckman).

Dry CYTODEX® beads (Sigma-Aldrich® # C3275) of 0.5 g are hydrated in 50 mL HYCLONE™ DPBS pH 7.4 (Fisher Scientific # SH3026402) for at least 3 hours at room temperature. The tube (Falcon # 352098) containing the beads is inverted to mix gently every 0.5 hour. Supernatant is discarded. Beads are washed three times with fresh PBS and re-suspended in 50 mL PBS to give ˜20,000 beads/mL. The bead suspension is autoclaved for 15 minutes at 115° C. and stored at 4° C. until use.

Beads are gently mixed and 0.5 mL (approximately 10,000 beads) suspension is transferred into a 50 mL tube (Falcon # 352098). Beads are washed twice in 10 mL of warm EBM-2 medium (Lonza # CC-3156) plus SINGLEQUOTS™ (Lonza # CC-4147). The medium is carefully removed after final wash. Washed beads are mixed with 8 million HUVEC cells in total volume of 20 mL. The tube containing beads and HUVEC cells is placed in an incubator at 37° C. with 5% CO₂ for 4 hours and gently mixed every 20 minutes by inverting the tube several times. After incubation, beads with HUVEC cells are transferred into a T25 flask (NUNC™ cat # 156499) and incubated at 37° C. with 5% CO₂ overnight.

Fibrinogen (Sigma #F4883) is dissolved in HYCLONE™ DPBS at 2 mg/mL. Aprotinin (Sigma #A3428) is added to the fibrinogen solution at a concentration of 0.15 units/mL and gently mixed. The solution is sterilized by filtering through a 0.22μ filter (EMD Millipore #SCGP00525) and used immediately.

The HUVEC-coated beads in the T25 flask is transferred to a 50 mL tube and washed twice using 10 mL of warm EBM-2 medium plus SINGLEQUOTS™ (Lonza #CC-4147). The medium is removed gently. HUVEC-coated beads (approximately 10,000) are re-suspended in 50 mL of sterilized fibrinogen solution with 2 million CAFs. Thrombin (Sigma #T4393) is reconstituted with sterile water to 50 units/ml. 0.6 unit (12 μl) of the thrombin solution is added per well of a 24-well plate (Cellvis #P24-1.5H-N), followed by the addition of 500 μl/well of the fibrinogen/beads/CAF solution. The solution is allowed for fibrin gel formation for 15 minutes at room temperature and then incubated at 37° C. with 5% CO₂ for one hour. 0.5 mL of warm EBM-2 medium plus SINGLEQUOTS™ (Lonza #CC-4147) is added on top of the fibrin gel in each well and replaced every 3 to 4 days until the end of the experiment.

For the neo-mode sprouting assay, test compound diluted in the indicated concentration is added to each well. Plates are incubated at 37° C. with 5% CO₂, and medium with test compound is changed every 3 to 4 days until the assay is completed.

For the established-mode sprouting assay, the method is the same as described above for the neo-mode sprouting assay, except the HUVEC-coated beads in the fibrin gel are cultured for 3 to 7 days before the addition of the test compound. The test compound treatment runs for 7 days. Medium with test compound is changed every 3 to 4 days until the assay is done.

After the assay is finished, plates are fixed in 0.5 mL of 4% paraformaldehyde (PFA, Electron Microscopy Sciences #15710) overnight at 4° C., washed once in PBS, perrneabilized with 0.5 mL of 0.5% TRITON™ X-100 (Sigma-Aldrich #T9284)/PBS for 10 minutes at 4° C. followed by washing three times in 100 mM glycine (BIO-RAD® #161-0718)/PBS at room temperature. The plates are blocked with 1 ml/well immunofluorescence (IF) buffer which contains 0.1% bovine serum albumin (BSA, GIBCO™ #15260-037), 0.2% TRITON™ X-100, 0.05% Tween®-20 (Thermo Scientific #28320) in PBS plus 10% goat serum (Invitrogen #16210). Endothelial cells are stained with sheep anti-human CD31 antibody (R&D Systems #BAF806) reconstituted in 500 mL PBS at 1:100 in IF buffer and 10% goat serum. α smooth muscle actin-positive cells are stained with anti-α smooth muscle actin antibody, Cy3 antibody (Sigma, # C6198) at 1:200 in IF buffer plus 10% goat serum. The staining solution is added to each well at 500 μL/well. The plates are kept at 4° C. overnight. The staining solution is removed on the following day, and plates are washed using 0.5 mL IF buffer three times. Secondary antibody ALEXA FLUOR® 488 Donkey anti-sheep IgG (H+L, Molecular Probes #A-11015) at 1:200 dilution in the IF buffer plus 10% goat serum is added for one hour incubation at room temperature. The plates are washed three times using IF buffer to remove any unbound secondary antibody. DAPI (4′,6-Diamidino-2-phenylindole dihydrochloriden. Invitrogen #D1306) at 5 mg/mL is diluted at 1:10000 in PBS and 0.5 mL is added to each well for one hour incubation at room temperature. The plates are washed twice with PBS and total length of CD31 positive endothelial sprouts and SMA-positive cells are imaged by scanning the plates on a CellInsight (ThermoFisher Scientific) instrument using the 2× objective. Image data are directly from the CellInsight (CD31, green; SMA, red) and numeric data are analyzed in JMP® (SAS Statistical Analysis Software).

N-(3 -fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide treatment reduced VEGF-A dependent and VEGF-A independent endothelial sprouting with statistical significance.

A modified in vitro co-culture angiogenesis assay as described in Mabry, R., et al., MAbs. 2010 January-February;2(1):20-34 and Nakatsu, M N, Meth Enzymol. 2008:443:65-82) using HUVECs and CAF cells is used to evaluate the effects of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide on endothelial cell sprouting. CAFs and cytodex beads coated with HUVECs are embedded into a fibrin gel to form endothelial sprouts that are covered with smooth muscle actin (SMA) positive pericytes. For a one-week period, the endothelial cells undergo a series of phenotypic changes that result in a stable interconnected network of endothelial sprouts that are covered with SMA-positive cells. Endothelial sprouting is dependent on CAF derived VEGF-A in the media for up to 7-10 days after which sprout elongation and stability is less dependent on VEGF-A.

Inhibition of VEGF-A signaling at the start of the assay (neo-mode, days 0-7) with ramucirumab (10 μg/ml) led to significant inhibition of endothelial sprouting (4-fold reduction). To mimic therapeutic inhibition of angiogenesis, treatment with ramucirumab is started on day 7 (established mode) after endothelial sprouts and pericyte coverage is formed. Treatment of preformed sprouts with ramucirumab (10 μg/ml) was less effective and only reduced endothelial sprouting 1.4 fold. Similar to VEGFR2 inhibition, addition of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide at the start of the assay (neo-mode, days 0-7) potently inhibited endothelial sprouting in a dose dependent manner. At the highest concentration tested, 300 nM, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide reduced endothelial sprouting 7.5 fold. Treatment of preformed sprouts (established mode), which are significantly less sensitive to VEGFR2 inhibition, with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, led to a significant reduction in endothelial sprouting (2.7 fold). These results demonstrate that N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide potently inhibits VEGF-A dependent and VEGF-A independent endothelial sprouting. The MET-specific inhibitor PF04217903 (LSN2900296) is less active in inhibiting endothelial sprouting when added at the beginning of the assay (neo-mode) where sprouting is VEGF-A dependent (days 0-7) and when added to preformed sprouts (established mode) that are less dependent on VEGF-A for sprout elongation and stability (days 7-14). PF04217903 is inactive in inhibiting endothelial sprouting. These data suggest the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is not MET dependent.

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide inhibited sprouting through day 14 while ramucirumab inhibited sprouting only up to day 7. Control (PF4217904) had no effect.

Ramucirumab is a specific VEGFR2 inhibitor and demonstrated endothelial sprouting reduction in this assay for the first 7 days when the sprouting is VEGF-A dependent. The MET specific inhibitor PF4217903 showed little or no effect in this assay throughout the 14 days of this assay, indicating that MET does not play a role in this vascular model, and MET is one of the oncokinase targets for N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide. The data from this assay suggest that one or more of the other targets of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, such as Tie2 (a.k.a. TEK), AXL, PDGFRA, and MERTK may cause inhibition of endothelial sprouting throughout the 14 days of this angiogenesis assay. Thus the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is different from ramucirumab and supports the combination of these two agents in cancer treatment to provide further treatment benefit.

### N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide Inhibits Cord Formation in Neo and Established Modes

A VEGF-A induced cord formation assay is performed in micro-titer plates according to Falcon et al., J Hematol Oncol. 2013;6:31. The assay is performed as the neo-mode (neoangiogenic adipose derived stein cells (ADSC) and human endothelial colony forming cells (ECFC) co-culture cord formation Assay) and the Established mode (established ADSC and ECFC co-culture cord formation assay).

For the neo-mode assay, the ADSC and ECFC are co-cultured with ANGIOKIT™ optimized media (Cell Systems Biology). ADSC are plated in 96-well plates at 40-50K cells per well in 100 μL and incubated overnight at 37° C., 5% CO₂. The next day, the media is removed and 4-5 K ECFC per well in 50-100 μL of media is plated on top of the ADSC monolayer and incubated at 37° C., 5% CO₂ for 3-6 hours before the addition of 20 ng/mL VEGF-A and test compounds. Co-cultures are grown for 7 days, at which time the cells are fixed, stained, and imaged in a scanning device. Cord area is quantified.

For the established mode assay, ADSC and ECFC co-culture are plated as described above for the neo-mode assay. After the ECFC are allowed to attach, 20 ng/mL VEGF-A is used to stimulate and to establish the cord network. On Day 4, the media is changed to contain fresh VEGF-A in the presence or absence of test compound at the indicated concentrations. After addition of the test compound, cultures are allowed to grow an additional 3-4 days before the cells are fixed, stained, and imaged as described above, to investigate network disruption or cord regression.

Ramucirumab has been shown to be effective in the neo-mode of this assay, (IC₅₀=0.48 μg/mL (S.D. 0.30, n=3) [0.48 μg/mL=3.2 nM], but not the established mode (IC₅₀>10 μg/mL). See Falcon et al., J Hematol Oncol. 2013;6:31. Sunitinib, a small molecule multi-kinase inhibitor with anti-VEGFR2 activity, has been shown to be effective in the neo-mode of this assay (IC₅₀=0.038 μM; S.D. 0.013, n=3), but not the established mode. See Falcon et al., J Hematol Oncol. 2013;6:31.

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide demonstrated potent activity in both the neo-mode (IC₅₀=0.013 μM; S.D. 0.007, n=5) and the stablished mode (IC₅₀=0.016 μM 0.011, n=4).

A control MET-specific inhibitor is evaluated in this assay. The control MET-specific inhibitor showed only a slight activity in the neo-mode (IC₅₀=5.61 μM; S.D. 1.80, n=2) and no activity in the established mode (IC₅₀>10 μM; n=2). This suggested that the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is not due to its activity for MET.

Ramucirumab is a specific VEGFR2 inhibitor and demonstrated reduction in this cord formation assay in the neo-mode when the cord formation was VEGF-A dependent. The MET specific inhibitor PF4217903 showed little or no effect in this assay both in the neo-mode or in the established mode, indicated that MET does not play a role in this vascular model and MET is one of the oncokinase targets for N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide. The data from this assay suggest that one more of the other targets of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, such as Tie2 (a.k.a. TEK), AXL, PDGFRA, and MERTK is involved in the inhibition of cord formation in neo-mode and in the established mode. Thus the anti-angiogenic activity of N-(3 -fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is different from ratnucirumab and supports the combination of these two agents in cancer treatment to provide further treatment benefit.

### Combination of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and DC101 in a Mouse Ear Vascular Model Induced by VEGF-A

The mouse ear vascular model for evaluating anti-angiogenic compounds is set up according to Nagy el al., Methods Enzymol. 2008;444:43-64. Blood vessels are induced in the mouse ear by VEGF-A (vascular endothelial growth factor A) via the injection of adenoviral vectors carrying the coding sequence of murine VEGF-A into the mouse ears.

DC101 is dosed at 40 mg/kg twice weekly via intraperitoneal injection. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is dosed at 12 mg/kg orally once daily. For the Day 5 results, the compounds or vehicle control are dosed daily from days 1-5. For the Day 20 results, the compounds or vehicle control are dosed daily from days 10-20. For Day 60 results, the compounds or vehicle are dosed daily from days 50-60. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is formulated as a solution in 10% polyethylene glycol (PEG) 400 in 90% of 20% CAPTISOL® in H₂O and prepared fresh each week of dosing. DC101 is diluted in PBS each week of dosing. Vehicle control is 10% PEG 400 in 90% of 20% CAPTISOL® in H₂O dosed orally once daily.

The effect of the combination treatment, vehicle control or each single agent wis evaluated and quantified with the expression of 50 different vascular markers using the QUANTIGENE® Plex 2.0 Assay kit according to the manufacturer's protocol (Affymetrix eBioscience).

Synergy is determined if the combination is significantly different from control, the effect size is large (combination-control and combination-expected additive response >1.0 or <−1.0), and the p-value for synergy is significant (<0.05). P-values are compared to vehicle control and are Bonferroni adjusted.

Markers that are synergistically affected by the combination treatment are late (day 60) and are markers more for pericytes than endothelium (Table 1). Markers of pericytes are Acta2, Cspg4 (NG2), Notch 1 and Notch 3 and their ligands (DLL1, DLL3, Jag2), and PDGFB. This is consistent in that the combination showed effect in reducing early blood vessels formation and in remodeling early and later blood vessels and in stabilizing normal blood vessels.

TABLE 3
Markers that were affected synergistically by the combination

| | | p-value\hat{ } | | | |
|---|---|---|---|---|---|
| | | | | DC101 + | Synergy |
| Gene | Day | DC101 | LY2801653 | LY2801653 | p-value |
| Normalized | | | | | |
| to 3 house- | | | | | |
| keeper genes | | | | | |
| Acta2 | 60 | 1 | 1 | 0.0092 | 0.013 |
| Igfbp3 | 5 | 0.127 | 0.405 | <0.0001 | <0.0001 |
| Mest | 5 | 0.0005 | 0.07 | <0.0001 | 0.003 |
| Normalized to | | | | | |
| PECAM-1 | | | | | |
| Cdh6 | 60 | 1 | 1 | 0.0009 | 0.013 |
| Cspg4 (NG2) | 60 | 0.21 | 0.11 | <0.0001 | 0.002 |
| DLL1 | 5 | 1 | 0.203 | <0.0001 | 0.008 |
| DLL3 | 60 | 1 | 1 | <0.0001 | 0.003 |
| Gng11 | 60 | 1 | 1 | 0.0007 | 0.001 |
| Hey2 | 60 | 1 | 1 | 0.003 | 0.007 |
| Jag2 | 60 | 0.065 | 0.08 | <0.0001 | 0.028 |
| Mlana | 60 | 0.74 | 1 | <0.0001 | 0.003 |
| Notch1 | 60 | 0.04 | 0.24 | <0.0001 | 0.006 |
| Notch3 | 60 | 0.19 | 0.21 | <0.0001 | 0.004 |
| PDGFA | 60 | 1 | 1 | <0.0001 | 0.003 |
| PDGFb | 60 | 1 | 1 | <0.0001 | 0.001 |
| Ptgds | 60 | 0.35 | 0.68 | <0.0001 | 0.037 |
| Sdc4 | 60 | 0.016 | 0.037 | <0.0001 | 0.019 |

\hat{ }P-values are compared to vehicle control and are Bonferroni adjusted.

Additivity is determined if the combination is significantly different from control, the effect size is large (combination-control and combination-expected additive response >1.0 or <−1.0), and one of the single agents is not significantly different from control and the p-value is not significant for the combination comparing to expected additive response. P-values are compared to vehicle control and are Bonferroni adjusted.

Markers that are not synergistically affected and affected additively by the combination are evenly distributed between early (day 5) and late (day 60) (Table 2). These markers were also an even mix of endothelial markers (e.g., CD34, PECAM1, vwf, PDGFRB, PDGFRA, VEGR2 and its ligands VEGFA) and pericyte markers (e.g., Acta2, Cspg4 (NG2), Notch 1 and Notch 3 and their ligands (DLL1, DLL3, Jag2)). These are consistent with the combination effect throughout the entire study period of days 5-60.

The combination of ramucirumab and N-(3-fluoro-4-(1-methyl-6-(1H-pyrazol-4-yl)-1H-indazol-5-yloxy)phenyl)-1-(4-fluorophenyl)-6-methyl-2-oxo-1 ,2-dihydropyridine-3-carboxamide results in synergistic anti-angiogenic activity in vivo in a mouse ear VEGF-driven angiogenesis model.

TABLE 4
Effect on Markers that were additive by the combination

| | | p-value\hat{ } | | |
|---|---|---|---|---|
| | | | | DC101 + |
| Gene | ay | DC101 | LY2801653 | LY2801653 |
| Normalized to 3 | | | | |
| housekeeper genes | | | | |
| CD34 | 0 | 0.29 | 0.42 | <0.0001 |
| Cdh5 | | 0.31 | 0.03 | <0.0001 |
| Cytl1 | | 0.06 | 0.06 | 0.0001 |
| DLL4 | | 0.23 | 0.97 | 0.0016 |
| DLL4 | 0 | 0.005 | 1 | <0.0001 |
| EphA1 | 0 | 0.03 | 0.23 | 0.0013 |
| Gng11 | | 0.22 | 0.17 | <0.0001 |
| VEGFR2 | | 0.35 | 0.51 | <0.0001 |
| Lor | 0 | 0.25 | 1 | 0.031 |
| Mest | 0 | 0.025 | 1 | 0.005 |
| Notch4 | | 0.07 | 1 | <0.0001 |
| Pdgfd | 0 | 0.013 | 0.34 | 0.0003 |
| PECAM1 | 0 | 0.008 | 0.18 | <0.0001 |
| Rgs5 | 0 | <0.0001 | 0.15 | <0.0001 |
| VEGFc | 0 | 0.74 | 1 | 0.011 |
| Vwf | | 0.78 | 0.35 | 0.0003 |
| Normalized to PECAM-1 | | | | |
| Acan | 5 | 0.03 | 1 | <0.0001 |
| Acan | 60 | 0.04 | 0.69 | <0.0001 |
| Aqp1 | 60 | 0.04 | 0.3 | <0.0001 |
| BMP4 | 60 | 0.02 | 0.64 | <0.0001 |
| Ccl24 | 5 | 0.008 | 0.37 | <0.0001 |
| Cdh6 | 5 | 0.07 | 0.41 | 0.002 |
| Col6a2 | 5 | 0.08 | 0.06 | <0.0001 |
| Col6a2 | 60 | 0.16 | 0.77 | <0.0001 |
| DLL1 | 60 | 0.17 | 0.18 | <0.0001 |
| Dpp4 | 60 | 0.02 | 0.63 | 0.0001 |
| Dtx1 | 5 | 0.51 | 0.001 | <0.0001 |
| Eif3f | 60 | 0.008 | 0.15 | <0.0001 |
| Jag1 | 5 | 0.25 | 0.24 | <0.0001 |
| Jag1 | 60 | 0.04 | 0.08 | <0.0001 |
| Jag2 | 5 | 0.033 | 0.064 | <0.0001 |
| Lef1 | 60 | 0.16 | 0.26 | <0.0001 |
| Lor | 60 | 0.024 | 0.32 | <0.0001 |
| Mest | 5 | 0.0009 | 1 | <0.0001 |
| Mlana | 5 | 0.003 | 0.06 | <0.0001 |
| PDGFa | 5 | 0.019 | 0.06 | <0.0001 |
| PDGFRa | 5 | 0.43 | 0.004 | <0.0001 |
| PDGFRb | 60 | 1 | 1 | 0.003 |
| Sdc4 | 5 | 0.018 | 0.09 | <0.0001 |
| Sp6 | 60 | 0.007 | 0.21 | <0.0001 |
| Tbp | 60 | 0.11 | 0.83 | <0.0001 |
| VEGFa | 5 | 0.19 | 0.75 | <0.0001 |
| VEGFa | 60 | 0.004 | 0.25 | <0.0001 |
| Vwf | 5 | 0.97 | 1 | 0.02 |
| Vwf | 60 | 0.06 | 0.07 | <0.0001 |

\hat{ }p-values are compared to vehicle control and are Bonferroni adjusted

### An Open-Label, Phase 1a/1b Study of Ramucirumab in Combination with Other Targeted Agents in Advanced Cancers: Study Arm 1: Ramucirumab plus Merestinib in Colorectal Cancer

Study Design

The study is a multicenter, nonrandomized, open-label Phase 1a/1b study evaluating the safety and efficacy of ramucirumab in combination with merestinib for the treatment of advanced or metastatic colorectal cancer. Phase 1a will consist of a dose-limiting toxicity (DLT) observation period. Phase 1b will consist of an expansion period. This study is designed to investigate ramucirumab in combination with merestinib which was chosen based on scientific rationale, prior clinical experience with other targeted agents, and experience with ramucirumab, to treat patients with various types and stages of cancer. The primary analysis will be conducted at the earlier of the following time points:
1. approximately 1 year after the last patient in the study receives his or her first dose of study treatment in Phase 1b; or
2. after all patients in the study have discontinued all study treatment.

Study Objectives and Endpoints

The primary objective of this study is to assess the safety and tolerability of ramucirumab plus merestinib in specific cancer indications, specifically advanced or metastatic colorectal cancer.

The secondary objectives of this study are 1) to assess the pharmacokinetics of ramucirumab and merestinib when co-administered, and 2) to document the preliminary antitumor activity observed with ramucirumab in combination with merestinib for example,
Proportion of Participants Who Exhibit Complete Response (CR) or Partial Response (PR) [Overall Response Rate (ORR)]
Progression Free Survival (PFS).

The exploratory objective of this study is to assess the relationship between biomarkers (including but not limited to biomarkers of the VEGF and cMET pathways) associated with treatment, treatment mechanism of action, cancer, and immune response to clinical outcome.

The primary endpoints of this study are 1) to identify the dose-limiting toxicity (DLT) or DLT-equivalent toxicity, and 2) to monitor the safety of the combination of ramucirumab plus merestinib, including clinical and laboratory significant events.

The secondary endpoints of this study are 1) to measure the minimum serum/plasma concentration of ramucirumab plus merestinib, and 2) to determine ORR per RECIST 1.1 (Eisenhauer et.al., 2009) for solid tumors.

Treatment Plan

All patients enrolled in this study will receive ramucirumab intravenously administered in combination with orally administered merestinib during Phase 1a (DLT Observation Period) and Phase 1b (Expansion Period). Phase 1a will last for one treatment cycle (28 days). Patients who complete Phase 1a without a DLT will continue treatment until a criteria for discontinuation is met.

Rainucirumab (8 mg/kg administered IV on Day 1 and Day 15 every 28 days) and merestinib (80 mg administered orally once per day) are administered in a 28-day treatment cycle during Phase 1a (DLT observation period) of the study. Doses of ramucirumab and/or merestinib may be delayed, omitted, or reduced if a patient experiences an adverse event described below or a DLT-equivalent toxicity (defined as a DLT occurring after the DLT observation period) using CTCAE Version 4.0 (NCI 2009) to assign adverse event terms and severity grades. After 1 treatment cycle of 28 days and completion of an interim safety analysis, continuation of ramucirumab and merestinib at the above doses may resume for Phase 1b (the expansion period) until a criterion for discontinuation is met.

The following criteria applies to maintaining a control on the enrolled population for the study but should not be construed to limit the potential population that is intended under this patent application.

Initially, 3 patients are enrolled in Phase 1a. Additional patients may be enrolled based on the following criteria.
If none of the initial 3 patients treated at a given dose level develops a DLT, Phase 1b will start.
If 1 of the initial 3 patients treated at a given dose level develops a DLT, 3 additional patients will be enrolled at that dose level.
If ≤1 of the 6 patients treated at a given dose level develops a DLT, Phase 1b will start.
If ≥2 patients treated at a given dose level develop a DLT, enroll in that study will stop, and an alternative dose level may be considered.

In Phase 1b, 15 additional patients are enrolled.

Inclusion Criteria

Patients are eligible to be included in this study if they meet all of the following criteria:
1) Have a diagnosis of the type of cancer described below and meet the following requirements:
   a) histopathologically confirmed advanced or metastatic colorectal cancer; excluding primary tumors of appendiceal origin
   b) have at least 1 measurable lesion assessable by radiological imaging. Tumor lesions located in a previously irradiated area are considered measureable if progression has been demonstrated in such lesions;
   c) have received prior second-line treatment with oxaliplatin and/or irinotecan, and no other licensed/standard-of-care therapies are available. If the participant has RAS wild type colorectal cancer, he or she also must have received prior treatment with an epidermal growth factor receptor monoclonal antibody;
   d) have not received previous systemic therapy (including investigational agents) targeting programmed cell death protein 1 (PD-1)/ PD-1 ligand (PDL 1) or PD-1/PDL-2 signaling pathways. Prior therapy with other immune checkpoint inhibitors, including but not limited to, anti-CD137 antibody or anticytotoxic T-lymphocyte-associated antigen-4 antibody, is not permitted;
2) have adequate organ function
3) are, in the judgment of the investigator, appropriate candidates for experimental therapy after available standard therapies have failed to provide clinical benefit;
4) have discontinued all previous treatments for cancer and recovered from the acute effects of therapy, other than less than or equal to Grade 2 neuropathy or nonserious and nonlife-threatening toxicities such as alopecia, altered taste, and nail changes;
5) have a performance status of 0 or 1 on the Eastern Cooperative Oncology Group scale;
6) are at least 18 years old at the time of screening;
7) men and women must agree to the use an effective method of contraception during the study and for at least 3 months post last dose of study drug administration. Women of child-bearing potential must have negative serum and urine pregnancy tests at screening and during each treatment cycle, respectively;
8) have given written informed consent prior to any study-specific procedure;
9) have a life expectancy greater than or equal to 3 months; and
10) are able to swallow tablets and/or capsules.

Exclusion Criteria

Patients will be excluded from the study if they meet any of the following criteria:
1) have a serious illness or medical condition including, but not limited to, the following: active or uncontrolled clinically serious infection; inadequate biliary drainage with evidence of unresolved biliary obstruction;
2) have prior or concurrent malignancies, inclusive of hematologic, primary brain tumor, sarcoma, and other solid tumors, unless in complete remission with no therapy for a minimum of 5 years (with exceptions for adequately treated non-melanomatous skin cancer and curatively treated cervical carcinoma in situ or other noninvasive carcinoma or in situ neoplasm),
3) have active gastrointestinal (GI) disease characterized by inflammatory bowel disease, malabsorption syndrome, or frequent Grade 2 or more diarrhea;
4) are pregnant or breastfeeding;
5) have previously documented brain metastases, leptomeningeal disease, or uncontrolled spinal cord compression;
6) have experienced any of the following: a major surgical procedure, significant traumatic injury, non-healing wound, peptic ulcer, or bone fracture less than or equal to 28 days prior to enrollment, or placement of a subcutaneous venous access device less than or equal to 7 days prior to the first dose of study treatment unless the procedure is of low risk of bleeding in the judgment of the investigator;
7) have an elective or a planned major surgery during the course of the trial;
8) have a known allergy or hypersensitivity reaction to any of the treatment components;
9) have uncontrolled hypertension;
10) have experienced any arterial thromboembolic event within 6 months prior to enrollment;
11) have experienced any Grade 3 or 4 venous thromboembolic event that is considered by the investigator to be life threatening or that is symptomatic and not adequately treated by anticoagulation therapy, within 6 months prior to enrollment;
12) have a history of GI perforation and/or fistulae within 6 months prior to enrollment
13) have experienced any bleeding episode considered life-threatening, or any Grade 3 or 4 GI/variceal bleeding episode in the 3 months prior to enrollment requiring transfusion or endoscopic or operative intervention; or
14) have congestive heart failure or poorly controlled cardiac arrhythmia per New York Heart Association Class II-IV heart disease.

Treatment with the combination may continue for up to 1 year (12 cycles). Patients receiving clinical benefit may continue treatment during continued access period. Doses of ramucirumab and/merestinib may be delayed, omitted, or reduced if the patient experiences an adverse event or a DLT-equivalent toxicity. In case of difficulty in assigning relatedness to one study drug or the other, the doses of both study drugs may be delayed, reduced, or omitted. Ratnucirumab dosing may be delayed for up to 28 days, and merestinib dosing may be omitted for up to 14 days. Treatment of either or both drug may be resumed at the dose prior to an adverse event or DLT-equivalent toxicity, or may be continued at a reduced dose.

Dose-Limiting Toxicities

Toxicity is considered dose-limiting if it is deemed at least possible related to either or both study drugs. A patient is considered evaluable for DLTs if he or she (1) receives at least 70% of the dose of the oral drug and completes the DLT observation period or (2) discontinues because of a DLT. Dose-limiting toxicities (DLTs) are defined as any of the following adverse events: Grade 4 thrombocytopenia (unless recovered in 24 hours and in the absence of bleeding) or Grade 3 thrombocytopenia complicated with Grade ≥2 bleeding, Grade 4 hematologic toxicity persisting >5 days, Grade ≥3 febrile neutropenia, Grade 3 nonhematologic toxicity that occurs despite maximal supportive medical management, and any other clinically significant toxicity deemed to be dose limiting, such as Grade 2 seizures or severe tremors. Exceptions may be made for: alopecia, nausea, vomiting, anorexia, diarrhea, or constipation that can be appropriately controlled and does not persist for >72 hours with treatment, asymptomatic electrolyte disturbance that can be treated with oral substitution therapy or by intravenous infusions requiring <24-hour hospitalization, and transient (≤5 days) Grade 3 elevations of hepatic transaminases ALT and/or AST without evidence of other hepatic injury in the setting of preexisting hepatic metastasis.

Ramucirumab Administration

Prior to each infusion of ramucirumab, premedicate all patients with an oral or intravenous histamine H1 antagonist, such as diphenhydramine hydrochloride. Additional premedication may be provided at the investigator's discretion. Ramucirumab infusions should be delivered in approximately 60 minutes. The infusion rate should not exceed 25 mg/min. Infusions >60 minutes are permitted in the following situations:
1) if needed in order to maintain an infusion rate ≤25 mg/min, or
2) if the patient previously experienced a ramucirumab IRR.

The actual dose of ramucirumab to be administered will be determined by measuring the patient's weight in kilograms at the beginning of each cycle. If the patient's weight fluctuates by more than ±10% from the weight used to calculate the prior dose, the dose must be recalculated. Recalculation of the ramucirumab dose for weight fluctuation of <10% is permitted, but not required.

Merestinib Administration

Merestinib is supplied as 40-mg tablets for oral administration and should be taken with at least 8 ounces (240 mL) of fluid at approximately the same time every day, with, or within 1 hour, of a meal consisting of at least 100 calories. However, on days when the patient receives both merestinib and ramucirumab, merestinib should be taken approximately 10 minutes prior to the start of ramucirumab infusion.

Discontinuation from Study Treatment

Patients will be discontinued from all study treatment (Phase 1a and Phase 1b) in the circumstances listed below. The reason for discontinuation and the date of discontinuation will be collected for all patients.
the patient is enrolled in any other clinical trial involving an investigational product or any other type of medical research judged not to be scientifically or medically compatible with this study;
the patient has progressive disease;
the patient becomes pregnant during the study;
the patient is significantly noncompliant with study procedures and/or treatment;
the patient, for any reason, requires treatment with another therapeutic agent that has been demonstrated to be effective for treatment of the study indication; discontinuation from study treatment will occur prior to introduction of the new agent;
the investigator decides that the patient should be discontinued from study treatment;
the patient requests to be discontinued from study treatment;
the patient's designee (for example, a parent, legal guardian, or caregiver) requests that the patient be discontinued from study treatment.

Study Assessments

In order to characterize the preliminary efficacy signal, tumor response rates/assessments are performed for each patient by radiological imaging and measurement of palpable or visible lesions per Response Evaluation Criteria in Solid Tumors, Version 1.1 (Eisenhauer et. al., 2009). For patients with solid tumors, computed tomography (CT) scans, including spiral CT, are the preferred methods of measurement (CT scan thickness recommended to be ≤5 mm). The CT portion of a positron emission tomography (PET)-CT scan may be used as a method of response assessment if the site can document that the CT is of identical diagnostic quality to a diagnostic CT. For solid tumors, a PET scan alone or as part of a PET-CT may be performed for additional analyses but may not be used to assess response. Tumor lesions located in a previously irradiated area are considered measureable if progression has been demonstrated in such lesions. Baseline imaging and measurement are defined by RECIST v1.1. Subsequent radiological imaging and measurement of palpable or visible lesions are performed according to RECIST v1.1 every 6 weeks (±7 days) for the first 6 months after enrollment and every 9 weeks (±7 days) thereafter until radiographic disease progression, death, or study completion, whichever occurs first.

The primary analysis will be conducted at the earlier of the following time points:
1. approximately 1 year after the last patient in that study receives his or her first dose of study treatment in Phase 1b; and
2. after all patients on that study arm have discontinued from all study treatment.

Tumor assessments are performed as scheduled even if study treatment is delayed or omitted, except when deemed not feasible due to the patient's clinical status. For patients who discontinue study treatment, tumor assessment and imaging may be performed every 9 to 12 weeks, depending on the standard of care, according to RECIST 1.1, by the same method used at baseline.

End Of Study

End of the study is the date of the last visit or last scheduled procedure for the last patient.

SEQUENCE LISTING

SEQ ID NO: 1
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIK
SEQ ID NO: 2
EVQLVQSGGGLVKPGGSLRLSCAASGFTFSSYSMNWVRQAPGKGLEWVSS
ISSSSSYIYYADSVKGRFTISRDNAKNSLYLQMNSLPAEDTAVYYCARVT
DAFDIWGQGTMVTVSS
SEQ ID NO: 3
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 4
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

1. A method of treating colorectal cancer in a patient, comprising administering to a patient in need of such treatment an effective amount of an antibody comprising a light chain variable region (LCVR) amino acid sequence of SEQ ID NO: 1 and a heavy chain variable region (HCVR) amino acid sequence of SEQ ID NO: 2, wherein the antibody binds to VEGFR2, and a compound which is merestinib, or a pharmaceutically acceptable salt thereof.

2. The method of claim 1, wherein the antibody comprises a light chain amino acid sequence of SEQ ID NO: 3, and a heavy chain amino acid sequence of SEQ ID NO: 4 and the antibody binds to VEGFR2.

3. The method of claim 2, wherein the antibody is ramucirumab.

4. The method of claim 1, wherein the compound or salt thereof is administered at a dose of about 40 mg to 120 mg once every day of a 28-day cycle.

5. The method of claim 3, wherein ramucirumab is administered on Days 1 and 15 of a 28-day cycle at a dose of about 4 mg/kg to about 12 mg/kg.

6. The method of claim 4, wherein the compound or salt thereof is administered at a dose of about 80 mg once every day of a 28-day cycle.

7. The method of claim 5, wherein the antibody is ramucirumab and ramucirumab is administered at a dose of about 8 mg/kg on Days 1 and 15 of a 28-day cycle.

8. The method of claim 1, wherein the antibody and the compound that is merestinib, or a pharmaceutically acceptable salt thereof, is administered simultaneously, separately, or sequentially.

9-11. (canceled)

12. The method of claim 6, wherein the compound or salt thereof is provided in the form of a tablet.

13. The method of claim 12, wherein the tablet is formulated by spray dried dispersion.

14-28. (canceled)

29. The method of claim 2, wherein the antibody and the compound that is merestinib, or a pharmaceutically acceptable salt thereof, is administered simultaneously, separately, or sequentially.

30. The method of claim 3, wherein the antibody and the compound that is merestinib, or a pharmaceutically acceptable salt thereof, is administered simultaneously, separately, or sequentially.

31. The method of claim 4, wherein the antibody and the compound that is merestinib, or a pharmaceutically acceptable salt thereof, is administered simultaneously, separately, or sequentially.

32. The method of claim 5, wherein the antibody and the compound that is merestinib, or a pharmaceutically acceptable salt thereof, is administered simultaneously, separately, or sequentially.

33. The method of claim 6, wherein the antibody and the compound that is merestinib, or a pharmaceutically acceptable salt thereof, is administered simultaneously, separately, or sequentially.

34. The method of claim 7, wherein the antibody and the compound that is merestinib, or a pharmaceutically acceptable salt thereof, is administered simultaneously, separately, or sequentially.

35. The method of claim 11, wherein the antibody and the compound that is merestinib, or a pharmaceutically acceptable salt thereof, is administered simultaneously, separately, or sequentially.

36. The method of claim 12, wherein the antibody and the compound that is merestinib, or a pharmaceutically acceptable salt thereof, is administered simultaneously, separately, or sequentially.
